(19) United States
(12) Patent Application Publication
(10) Pub. No.: US 2016/0090337 A1
(43) Pub. Date: Mar. 31, 2016

(54) PARAFFIN DEHYDROGENATION WITH OXIDATIVE REHEAT

(71) Applicant: UOP LLC, Des Plaines, IL (US)

(72) Inventors: Bryan J. Egolf, Crystal Lake, IL (US); Rajeswar Gattupalli, Arlington Heights, IL (US); Bipin V. Vora, Naperville, IL (US); Charles M. Brabson, Humble, TX (US); J.W. Adriaan Sachtler, Des Plaines, IL (US); Joseph E. Zimmermann, Arlington Heights, IL (US); David N. Myers, Hoffman Estates, IL (US)

(21) Appl. No.: 14/501,385

(22) Filed: Sep. 30, 2014

(51) Int. Cl. C07C 5/333; C07C 7/04

(52) U.S. Cl. CPC C07C 5/3337; C07C 7/04; C07C 2523/62; C07C 2527/138

(57) ABSTRACT

A process is presented for the dehydrogenation of paraffins. The process utilizes the combustion of a fuel within the dehydrogenation reactor to provide the heat of reaction for dehydrogenation. The process controls the combustion through limiting the oxidant concentration. A paraffin feedstream is mixed with a fuel, and the fuel/paraffin feedstream is mixed with an oxidant stream at the inlet of each dehydrogenation reactor.

### FIELD OF THE INVENTION

The field of this invention relates to the dehydrogenation of paraffins in multiple reaction zones.

### BACKGROUND

The dehydrogenation of paraffins is an important commercial hydrocarbon conversion process because of the existing and growing demand for olefins for the manufacture of various chemical products such as detergents, high octane gasolines, oxygenated gasoline blending components, pharmaceutical products, plastics, synthetic rubbers, and other products which are well known to those skilled in the art. One example of this process is the dehydrogenation of propane to produce propylene which can be polymerized to polypropylene, a common plastic.

Those skilled in the art of paraffin conversion processing are well versed in the production of olefins by means of catalytic dehydrogenation of paraffinic hydrocarbons. In addition, many patents have issued which teach and discuss the dehydrogenation of hydrocarbons in general. For example, U.S. Pat. No. 4,430,517 (Imai et al) discusses a dehydrogenation process and catalyst for use therein.

Most catalysts for the dehydrogenation of hydrocarbons are susceptible to deactivation over time. Deactivation will typically occur because of an accumulation of deposits that block active pore sites or catalytic sites on the catalyst surface. Where the accumulation of coke deposits causes the deactivation, reconditioning the catalyst to remove coke deposits restores the activity of the catalyst. Coke is normally removed from the catalyst by contact of the coke-containing catalyst with an oxygen-containing gas at a high enough temperature to combust or remove the coke in a regeneration process. In a moving bed process, the regeneration process is carried out by removing catalyst from the vessel in which the hydrocarbon conversion is taking place and transporting the catalyst to a separate regeneration zone for coke removal. Arrangements for continuously or semi-continuously removing catalyst particles from a bed in a reaction zone for coke removal in a regeneration zone are well known. U.S. Pat. No. 3,652,231 describes a continuous catalyst regeneration process which is used in conjunction with the catalytic reforming of hydrocarbons, the teachings of which are hereby incorporated by reference. In the reaction zone of U.S. Pat. No. 3,652,231, the catalyst is transferred under gravity flow by removing catalyst from the bottom of the reaction zone and adding catalyst to the top while reactants flow cross currently through a radial flow bed.

While technology has improved in the production of olefins through dehydrogenation processes, there is still room for improving the economics and the process to increase production and decrease cost. The most direct way to overcome the problems of space velocity limitations is to add more catalyst to the process. Increasing the catalyst volume is readily accomplished in the design stage for a new unit. Unfortunately for existing units, adding additional catalyst could require expensive modification or replacement of all of the reactors and the associated piping for the delivery of reactants and the transfer of catalyst between the reactors.

### SUMMARY

The present invention provides for a streamlined process that enables a reduction in equipment and space for performing the dehydrogenation of paraffins.

A first embodiment of the invention is a process for the dehydrogenation of paraffins comprising passing a feedstream comprising a paraffin and a fuel to a first dehydrogenation reactor unit in a series of at least two reactor beds, wherein each reactor bed is operated at dehydrogenation reaction conditions, and wherein the dehydrogenation reaction conditions include a catalyst, wherein the feedstream is preheated to at least 450 C, and preferably heated to at least 500 C; passing an oxidant feedstream comprising an oxidant to the first dehydrogenation reactor unit, adding a diluents to the oxidant stream, wherein the diluent comprises a light hydrocarbon, or a portion of the feedstream, and combusting the fuel and the oxidant in the first dehydrogenation reactor unit to raise the temperature of the first dehydrogenation reactor unit above 580 C and reacting the paraffin over the catalyst, thereby generating a first effluent stream comprising paraffins, olefins and hydrogen; and passing the first effluent stream and a second oxidant feedstream to a second dehydrogenation reactor unit and combusting the fuel and oxidant in the second dehydrogenation reactor unit to generate a second effluent stream. An embodiment of the invention is one, any or all of prior embodiments in this paragraph up through the first embodiment in this paragraph wherein the dehydrogenation reactor unit comprises a plurality of reactor beds, wherein each reactor bed includes an inlet for admitting a fresh stream of oxidant. An embodiment of the invention is one, any or all of prior embodiments in this paragraph up through the first embodiment in this paragraph further comprising adding a diluent to the oxidant feedstream. An embodiment of the invention is one, any or all of prior embodiments in this paragraph up through the first embodiment in this paragraph wherein the diluents is steam, a portion of the paraffin feedstream, or combination thereof. An embodiment of the invention is one, any or all of prior embodiments in this paragraph up through the first embodiment in this paragraph wherein the paraffin in the feedstream is a propane feedstream. An embodiment of the invention is one, any or all of prior embodiments in this paragraph up through the first embodiment in this paragraph wherein the paraffin in the feedstream is a butane feedstream. An embodiment of the invention is one, any or all of prior embodiments in this paragraph up through the first embodiment in this paragraph wherein the fuel in the feedstream is hydrogen. An embodiment of the invention is one, any or all of prior embodiments in this paragraph up through the first embodiment in this paragraph wherein the dehydrogenation reaction conditions include contacting the paraffin with a dehydrogenation catalyst at an elevated temperature, wherein the catalyst comprises a Group VIII metal on a support. An embodiment of the invention is one, any or all of prior embodiments in this paragraph up through the first embodiment in this paragraph wherein the series of reactor beds includes four reactor beds. An embodiment of the invention is one, any or all of prior embodiments in this paragraph up through the first embodiment in this paragraph wherein each reactor bed includes an oxidant feedstream at each reactor bed inlet. An embodiment of the invention is one, any or all of prior embodiments in this paragraph up through the first embodiment in this paragraph wherein the oxidant feedstream to each reactor bed is sufficient to combust the fuel and heat the feedstream sufficiently to provide for the effluent stream exiting at a temperature of at least 500 C. An embodiment of the invention is one, any or all of prior embodiments in this paragraph up through the first embodiment in this paragraph wherein the reaction conditions includes a pressure less than 450 kPa (absolute). An embodiment of the invention is one, any or all of prior embodiments in this paragraph up through the first embodiment in this paragraph wherein the reaction conditions include a pressure less than 250 kPa (absolute).

A second embodiment of the invention is a process for the oxidative dehydrogenation of paraffins comprising heating a paraffin feedstream mixed with a fuel to form a mixed feedstream; passing the mixed feedstream to the first dehydrogenation reactor unit in a plurality of dehydrogenation reactor units arranged in a series formation; splitting an oxidant feedstream into a plurality of portions and passing each portion to one of the plurality of dehydrogenation reactor units; adding a diluent to the oxidant stream, wherein the diluent comprises light hydrocarbons, or a portion of the feedstream; and combusting the fuel and oxidant in the reactor units to generate heat and dehydrogenating the paraffin over a dehydrogenation catalyst to generate a process stream from each of the reactor units comprising olefins and paraffins. An embodiment of the invention is one, any or all of prior embodiments in this paragraph up through the second embodiment in this paragraph further comprising passing the process stream from the last dehydrogenation unit to a fractionation unit to generate an olefins product stream and a paraffin recycle stream. An embodiment of the invention is one, any or all of prior embodiments in this paragraph up through the second embodiment in this paragraph further comprising combining the paraffin recycle stream with the paraffin feedstream. An embodiment of the invention is one, any or all of prior embodiments in this paragraph up through the second embodiment in this paragraph wherein the fuel is hydrogen. An embodiment of the invention is one, any or all of prior embodiments in this paragraph up through the second embodiment in this paragraph wherein the oxidant is selected from the group consisting of oxygen, an oxygen enriched stream, and mixtures thereof. An embodiment of the invention is one, any or all of prior embodiments in this paragraph up through the second embodiment in this paragraph wherein the paraffin is propane or butane. An embodiment of the invention is one, any or all of prior embodiments in this paragraph up through the second embodiment in this paragraph wherein the oxidant concentration of the combined oxidant and feedstream is less than 19% by volume.

Other objects, advantages and applications of the present invention will become apparent to those skilled in the art from the following detailed description and drawings.

### BRIEF DESCRIPTION OF THE DRAWING

FIG. 1 shows the flow for the dehydrogenation reactor system having four reactors; and

FIG. 2 shows the process with the dehydrogenation reactor system within the process flow scheme including treatment of the process stream and product recovery.

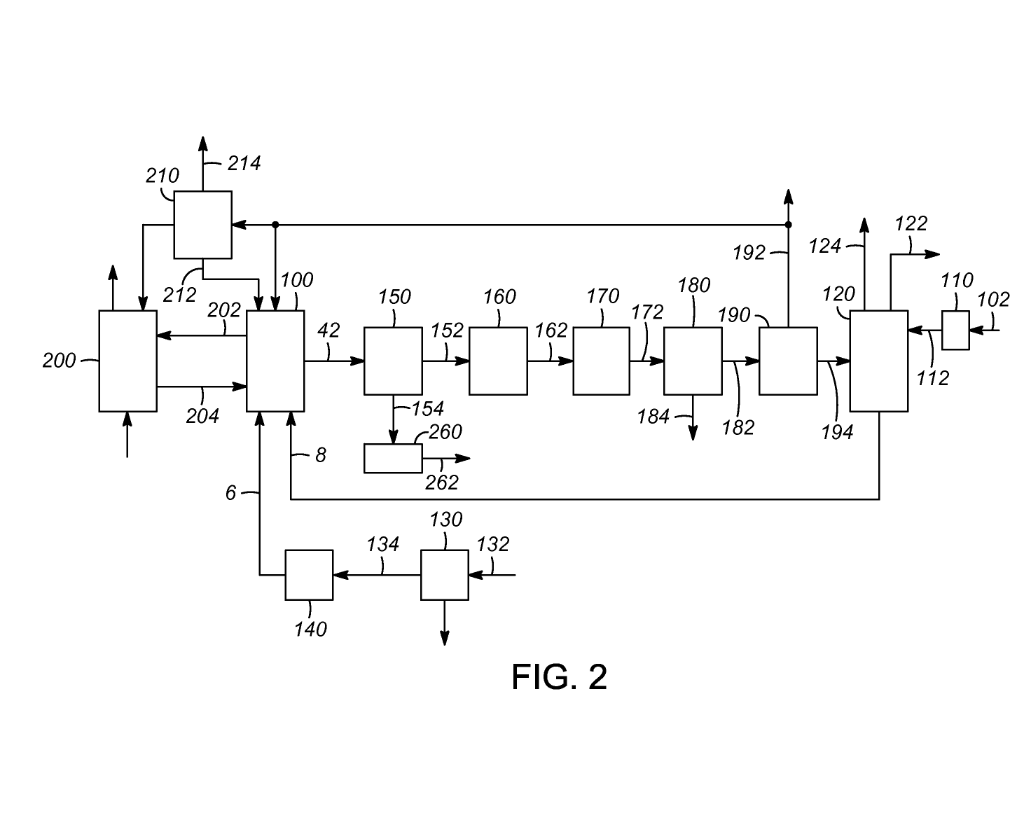

### DETAILED DESCRIPTION

Dehydrogenation processes are important sources for the conversion of paraffins to olefins. In particular the conversion of a light paraffin stream to a light olefin stream. One such example is the conversion of propane to propylene. The dehydrogenation process is an endothermic process, and the reactors and/or the feeds need to be heated to maintain the reaction. The current method of overcoming the self quenching reaction is to provide for multiple reactors with heating units, either fired heaters or other means, to reheat the process streams to temperatures for the reaction to continue.

A drawback for adding multiple reactors, with more heaters is the space needed to accommodate the equipment. To improve the ability to expand the process, one needs to be able to fit in additional equipment without added space, or to replace equipment, or to reduce the amount of equipment for the process.

The present invention provides for one method of improving the process of managing the heating requirements is a dehydrogenation process with an oxidative reheat. The dehydrogenation process injects a fuel and oxidant into the process, wherein the fuel and oxidant react to generate then compensating heat for the reaction. This reduces the need for the inter-reactor heaters within an existing plant, or for a future designed plant. By removing the inter-reactor heaters, space, or real estate, is freed up, and the reactors can be redesigned with a reduced area layout and reduced piping considerations. A benefit of the present design is the natural stacking of the reactor beds in a vertical stack with a series configuration for the flow of catalyst and process stream. An additional benefit of the present invention, is with the removal of the inter-reactor heaters, the pressure drop for the process is reduced, and allows for the operation at a lower pressure.

The fuel is preferably hydrogen, and is generated by the dehydrogenation process. The oxidant, preferably O2, is mixed with the inter-reactor effluent stream at the inlet to the subsequent reactor unit. The hydrogen is supplied by the prior reactor in the series. The hydrogen, H2, preferably burns, and provides the heat for the reaction for the paraffin dehydrogenation. This also reduces hot residence times in fired heaters and transfer lines, thereby reducing the possibility of undesired side reactions, such as cracking of the paraffin. The partial pressure of the oxidant can be controlled by the addition of steam, or another diluent. The paraffin can also be used as a diluent for this process.

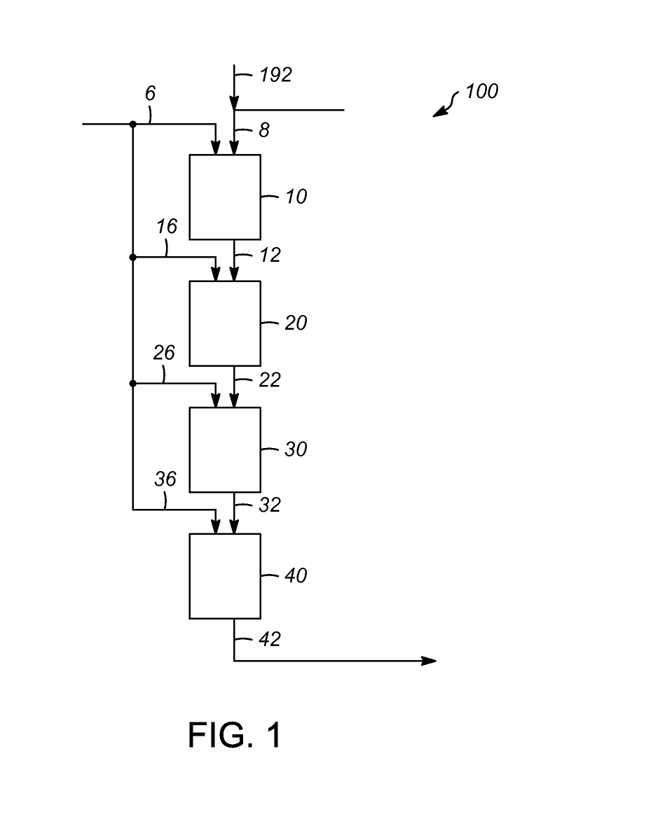

The present invention, as shown in FIG. 1, includes passing a feedstream 8 having a paraffin and a fuel to a first dehydrogenation reactor unit 10. The dehydrogenation reactor unit 10 comprises a catalyst, and is operated at dehydrogenation reaction conditions to generate an effluent stream 12 comprising olefins, paraffins and hydrogen. An oxidant stream 6 is passed to the first dehydrogenation reactor 10 and combusts the fuel to generate heat. The amount of oxidant and fuel is set to raise the temperature in the reactor unit 10 to a temperature between 540° C. and 700° C. The effluent stream 12 is passed to a second dehydrogenation reactor unit 20. A second oxidant stream 16 is passed to the second dehydrogenation reactor unit 20 to combust fuel in the effluent stream 12 in the second reactor unit 20, and to raise the temperature to between 540° C. and 700° C. The second reactor unit 20 generates a second effluent stream 22.

An exemplary number of reactor units is four, as represented by 100, but can include 3 or more reactor units. With four reactor units, the process continues with the second effluent stream 22 passed to a third reactor unit 30 and a third oxidant stream 26 is passed to the third reactor unit 30. The oxidant combusts with the fuel in the third reactor unit 30 to raise the temperature to between 540° C. and 700° C. The third reactor unit 30 generates a third effluent stream 32. The third effluent stream 32 passed to a fourth reactor unit 40 and a fourth oxidant stream 36 is passed to the fourth reactor unit 40. The oxidant combusts with the fuel in the fourth reactor unit 40 to raise the temperature to between 540° C. and 700° C. The fourth reactor unit 40 generates a fourth effluent stream 42. The reactor units can be stacked with the reactants and catalyst flowing from one reactor unit to another. The reactor units can also be fixed bed reactors, and can be positioned in a side by side orientation. The reactor units are arranged in a series format, and can be positioned in any convenient manner, in particular in a manner that facilitates the transfer of reactants between reactor units, and provides for access to admit flows or withdraw process streams.

The fourth effluent stream 42, or the final effluent stream from the series of reactor units, is passed to a product separation and recovery unit to generate an olefin stream and a paraffin stream. The paraffin stream can be recycled to the first reactor unit 10.

A preferred fuel is hydrogen, and with hydrogen generated by the process, the fuel is generated as the process stream passes from one reactor unit to the subsequent reactor unit within the series of reactor units.

The present invention can utilize fixed bed reactors or moving bed reactors. A preferred mode is for the use of moving bed reactors, with fresh regenerated catalyst passed to the first reactor unit. The catalyst from the first reactor unit is passed to the second reactor unit, and the catalyst continues flowing through the series of reactor units until the last reactor unit. The catalyst exiting the last reactor unit is sent to a regeneration unit, where the catalyst is regenerated and recycled to the first reactor unit.

The process can further include reactor units which comprise a plurality of reactor beds. In this embodiment, each reactor bed within each reactor unit has an inlet for the admission of a fresh stream of oxidant, and the oxidant stream to each reactor unit is split into a plurality of portions with each portion fed to a separate reactor bed.

The process can further comprise adding a diluent to the oxidant feedstream. The addition of a diluent provides control to be outside the flammability envelop for the mixture of oxidant, fuel and paraffin, while allowing the fuel and oxidant to be within an envelope to catalytically combust the fuel. The diluent, while not intended to be limiting, can comprise steam or the paraffin to be dehydrogenated. While the process of adding a diluent is known, the usual diluent is steam, or an inert component, as seen in U.S. Pat. Nos. 4,435,607 and 4,565,898. The use of steam alone, or another inert component present separation problems, and the amount of steam used can be energy intensive for removing downstream. The present process utilizes a light hydrocarbon, preferably C1-C5, that can act as a diluents. The preferred light hydrocarbon for a diluent is a portion of the paraffin from the feedstream. This allows for a reduction in the amount of steam used as a diluent, thereby reducing separation costs, especially if no supplemental steam is used. In addition, a benefit when the diluent is the paraffin, then there is no additional separation costs, and a portion of the diluent can even be dehydrogenated to be a part of the product stream.

The process of the present invention can be used for different paraffin streams, and is preferably operated at conditions such that the paraffin is in the vapor phase. A preferred paraffin feedstream is propane or butane.

Operating conditions for the preferred dehydrogenation zone, comprising the dehydrogenation reactor units, of this invention will usually include an operating temperature in the range of from 500° C. to 700° C., an operating pressure from 100 to 450 kPa (absolute) and a liquid hourly space velocity of from about 0.5 to about 50 for each catalyst bed. The preferred operating temperature will be within the range of from about 540° C. to 660° C., and the preferred operating pressure is 100 to 250 kPa (absolute). A more preferred operating conditions include a temperature is 580° C. to 645° C., an operating pressure from 100 to 170 kPa (absolute), and preferably operating conditions such that the effluent stream from each reactor unit is at a temperature of above 500° C., and most preferably at 580° C., with an operating temperature between 600° C. to 645° C. The temperature can be controlled by the flow of oxidant to the reactor units. When the effluent stream temperature is too high, the oxidant can be used as a quench to bring the inlet temperature of the feed and oxidant to the next reactor to below 580° C.

The feedstream comprising fuel and paraffin has a molar ratio from 0 to 1, with a preferred ratio between 0.1 and 0.7, and a more preferred ratio between 0.2 and 0.5.

The oxidative reheat conditions include mixing the oxidant stream with the feedstream, or with subsequent reactor units mixing the oxidant stream with the effluent stream from the previous reactor unit, using static mixers positioned at the inlet to the reactor units.

The preferred dehydrogenation catalyst is comprised of a Group VIII metal, and preferably a platinum group component, preferably platinum, a tin component and an alkali metal component with a porous inorganic carrier material. Another metal that can be used is chromium. Other catalytic compositions may be used within this zone if desired. The preferred catalyst contains an alkali metal component chosen from cesium, rubidium, potassium, sodium and lithium. The preferred alkali metal is normally chosen from cesium and potassium. Preferred dehydrogenation catalysts comprise an alkali metal and a halogen such as potassium and chlorine in addition to the tin and platinum group components. The preparation and use of dehydrogenation catalysts is well known to those skilled in the art and further details as to suitable catalyst compositions are available in patents, such as those cited above, and other standard references (U.S. Pat. Nos. 4,486,547 and 4,438,288).

The reactor system 100 of the present invention is a part of the full process for converting a paraffin stream to olefins. A paraffin stream 102 is treated in a feed treatment unit 110, to generate a treated feed 112. The treated feed 112 is passed through the product separation unit 120 and generates a feedstream 8 made up of recycled paraffin and new paraffin. The product separation unit includes a cold-box separator and fractionation section. The product stream is passed through a selective hydrogenation unit to selectively hydrogenate any acetylenes and diolefins. Air 132 is enriched in an air separation unit 130 to generate an oxygen stream 134 and a nitrogen stream 136. The oxygen stream can be enriched to a desired purity. The oxygen stream 132 is heated in a fired heater 140 to generate the heated oxidant stream 6.

The reaction system 100, generates a product stream 42 comprising the olefins. The product stream 42 is passed to a low pressure separator 150 to generate a process stream 152 with water removed and a waste water stream 154. The waste water stream 154 is passed to a waste water treatment unit 260 to recover stream 262. The process stream 152 is compressed with compressor 160 to form a compressed stream 162. The compressed stream 162 is passed to a chloride treater 170 to remove chlorides and generate a stream 172 of reduced chloride content. The stream 172 is passed to an acid gas treater 180 to remove acid gases 184 and generate a treated stream 182. An acid gas treater is an amine unit. The treated stream 182 is passed to a unit 190 to separate and recover hydrogen 192 for recycle and a process stream 194 comprising olefins. The hydrogen 192 is mixed with the paraffin feed 8 and fed to the dehydrogenation reactor system 100. The process stream 194 is separated in the separation unit 120 to generate the product stream 122 of olefins, and other hydrocarbons 124. The unconverted paraffins are passed to the feedstream 8 and recycled to the dehydrogenation reactor system 100.

An exemplary system includes a moving bed reactor system, and the catalyst in the system flows through the reactor system 100, and spent catalyst stream 202 is passed to a regenerator 200 with the regenerated catalyst stream 204 returned to the first reactor in the reactor system. Air 206 is passed to the regenerator 200 when carbon is burned off and is passed out as a flue gas stream 208. The recycle hydrogen 192 can be purified in a pressure swing absorber 210 to provide a stream 212 of higher concentration hydrogen for the process, and excess hydrogen 214 can be passed to other process units within a chemical plant.

Oxidative reheat that occurs directly in the reactor requires control of the oxidant. The process generally will require the oxygen concentration to be diluted to less than 6 vol. % prior to injection. This is managed with the addition of a diluent, and control of the amount of oxygen to be mixed with the feedstream. Hydrogen has a wide flammability envelope, and therefore, the oxygen concentration needs to be controlled to the limiting oxygen concentration. However, the hydrocarbons, in particular propane and propylene, have much higher limiting oxygen concentrations (LOC). The LOC at ambient is approximately 19 vol. %, and decreases to about 11 vol. % at temperatures of 500° C. By mixing the oxidant first into this stream rather than directly into the hydrogen containing process stream, elevated concentrations of oxygen can be obtained which can greatly minimize the need for managing inert diluent. This is further controlled by splitting the oxygen feed and passing a portion to the inlets at each of the reactor units in the series.

For the specific embodiment of oxygen as the oxidant, and steam as a diluent, the oxygen to steam molar ratio will be between 2:98 and 40:60, with the preferred range begin between 10:90 and 20:80.

A second observation is that the environment which a flammable mixture is exposed to has a significant impact on its flammability. For example, low temperatures and narrow channels limit the ability of a hydrogen stream to propagate a flame. This leads to static mixers having narrow channels, and injecting the oxidant stream into a structure media with narrow channels, such as a packed bed, microchannel distributor, etc., further providing control over undesired side reactions and flammability concerns.

While the invention has been described with what are presently considered the preferred embodiments, it is to be understood that the invention is not limited to the disclosed embodiments, but it is intended to cover various modifications and equivalent arrangements included within the scope of the appended claims.

What is claimed is:

1. A process for the dehydrogenation of paraffins comprising:
passing a feedstream comprising a paraffin and a fuel to a first dehydrogenation reactor unit in a series of at least two reactor beds, wherein each reactor bed is operated at dehydrogenation reaction conditions, and wherein the dehydrogenation reaction conditions include a catalyst, wherein the feedstream is preheated to at least 450 C;
passing an oxidant feedstream comprising an oxidant to the first dehydrogenation reactor unit, adding a diluents to the oxidant stream, wherein the diluents comprises a light hydrocarbon, or a portion of the feedstream, and combusting the fuel and the oxidant in the first dehydrogenation reactor unit to raise the temperature of the first dehydrogenation reactor unit above 580 C and reacting the paraffin over the catalyst, thereby generating a first effluent stream comprising paraffins, olefins and hydrogen; and
passing the first effluent stream and a second oxidant feedstream to a second dehydrogenation reactor unit and combusting the fuel and oxidant in the second dehydrogenation reactor unit to generate a second effluent stream.

2. The process of claim 1 wherein the dehydrogenation reactor unit comprises a plurality of reactor beds, wherein each reactor bed includes an inlet for admitting a fresh stream of oxidant.

3. The process of claim 1 further comprising adding a second diluent to the oxidant feedstream.

4. The process of claim 3 wherein the diluent is steam, a portion of the paraffin feedstream, or a combination thereof.

5. The process of claim 1 wherein the paraffin in the feedstream is propane.

6. The process of claim 1 wherein the paraffin in the feedstream is butane.

7. The process of claim 1 wherein the fuel in the feedstream is hydrogen.

8. The process of claim 1 wherein the dehydrogenation reaction conditions include contacting the paraffin with a dehydrogenation catalyst at an elevated temperature, wherein the catalyst comprises a Group VIII metal on a support.

9. The process of claim 1 wherein the series of reactor beds includes four reactor beds.

10. The process of claim 9 wherein each reactor bed includes an oxidant feedstream at each reactor bed inlet.

11. The process of claim 1 wherein the oxidant feedstream to each reactor bed is sufficient to combust the fuel and heat the feedstream sufficiently to provide for the effluent stream exiting at a temperature of at least 500° C.

12. The process of claim 1 wherein the reaction conditions includes a pressure less than 450 kPa (absolute).

13. The process of claim 12 wherein the reaction conditions include a pressure less than 250 kPa (absolute).

14. A process for the oxidative dehydrogenation of paraffins comprising:
heating a paraffin feedstream mixed with a fuel to form a mixed feedstream;
passing the mixed feedstream to the first dehydrogenation reactor unit in a plurality of dehydrogenation reactor units arranged in a series formation;
splitting an oxidant feedstream into a plurality of portions and passing each portion to one of the plurality of dehydrogenation reactor units;
adding a diluents to the oxidant stream, wherein the diluents comprises a light hydrocarbon, or a portion of the feedstream; and
combusting the fuel and oxidant in the reactor units to generate heat and dehydrogenating the paraffin over a dehydrogenation catalyst to generate a process stream from each of the reactor units comprising olefins and paraffins.

15. The process of claim 14 further comprising:
passing the process stream from the last dehydrogenation unit to a fractionation unit to generate an olefins product stream and a paraffin recycle stream.

16. The process of claim 15 further comprising combining the paraffin recycle stream with the paraffin feedstream.

17. The process of claim 14 wherein the fuel is hydrogen.

18. The process of claim 14 wherein the oxidant is selected from the group consisting of oxygen, an oxygen enriched stream and mixtures thereof.

19. The process of claim 14 wherein the paraffin is propane or butane.

20. The process of 14 wherein the oxidant concentration of the combined oxidant and feedstream is less than 19% by volume.
